SEQUENCE LISTING (17 sequences)

SEQ ID NO: 1 - DNA, 20 bp, Artificial Sequence
  Description of Artificial Sequence Primer
attagcggatcctacctgac

SEQ ID NO: 2 - DNA, 43 bp, Artificial Sequence
  Description of Artificial Sequence Primer
ccattgtgctgaatgttcatgtgtgctatggctatagaacggc

SEQ ID NO: 3 - DNA, 46 bp, Artificial Sequence
  Description of Artificial Sequence Primer
ggtgccgttctatagccatagcacacatgaacattcagcacaatgg

SEQ ID NO: 4 - DNA, 37 bp, Artificial Sequence
  Description of Artificial Sequence Primer
ttattcttctagatcactttatagttccccaaagaac

SEQ ID NO: 5 - DNA, 22 bp, Artificial Sequence
  Description of Artificial Sequence Primer
ctatgccatagcatttttatcc

SEQ ID NO: 6 - DNA, 17 bp, Artificial Sequence
  Description of Artificial Sequence Primer
agccaagctggagaccg

SEQ ID NO: 7 - DNA, 44 bp, Artificial Sequence
  Description of Artificial Sequence Primer
gggctaacaggaggaagcttccatgacacatgaacattcagcac

SEQ ID NO: 8 - DNA, 30 bp, Artificial Sequence
  Description of Artificial Sequence Primer
tgtgtcatggaagcttcctcctgttagccc

SEQ ID NO: 9 - protein, 245 aa, Artificial Sequence
  Description of Artificial Sequence Amino acid sequence of the truncated lysostaphin protein encoded by pJSB28
MTHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 10 - DNA, 735 bp, Artificial Sequence
  Description of Artificial Sequence Nucleotide sequence encoding the truncated lysostaphin protein in pJSB28
atgacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaag

SEQ ID NO: 11 - protein, 262 aa, Artificial Sequence
  Description of Artificial Sequence Amino acid sequence of the truncated lysostaphin protein and signal polypeptide encoded by pJSB20
MKKLLFAIPLVVPFYSHSTHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 12 - DNA, 786 bp, Artificial Sequence
  Description of Artificial Sequence Nucleotide sequence encoding the truncated lysostaphin protein and signal polypeptide in pJSB20
atgaaaaaactgctgttcgcgattccgctggtggtgccgttctatagccatagcacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaag

SEQ ID NO: 13 - protein, 244 aa, Artificial Sequence
  Description of Artificial Sequence Amino acid sequence of the truncated lysostaphin found in the plasmid pLss12F
THEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 14 - DNA, 732 bp, Artificial Sequence
  Description of Artificial Sequence Nucleotide sequence encoding the truncated lysostaphin found in the plasmid pLss12F
acacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataacgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaag

SEQ ID NO: 15 - protein, 247 aa, Artificial Sequence
  Description of Artificial Sequence Illustrative protein sequence
RAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 16 - protein, 248 aa, Artificial Sequence
  Description of Artificial Sequence Illustrative protein sequence
LRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 17 - protein, 246 aa, Artificial Sequence
  Description of Artificial Sequence Illustrative protein sequence
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

(19) United States
(12) Patent Application Publication
(10) Pub. No.: US 2005/0118159 A1
(43) Pub. Date: Jun. 2, 2005

(54) Truncated lysostaphin molecule with enhanced staphylolytic activity

(76) Inventors: Jeffrey R. Stinson, Brookeville, MD (US); Liouboy Grinberg, Gaithersburg, MD (US); James J. Mond, Silver Springs, MD (US); John F. Kokai-Kun, Frederick, MD (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/499,690

(22) PCT Filed: Dec. 23, 2002
(86) PCT No.: PCT/US02/40924

Related U.S. Application Data
(60) Provisional application No. 60/341,804, filed on Dec. 21, 2001.

(51) Int. Cl. A61K 38/48; C07H 21/04; C12N 9/48; C12N 15/74

(52) U.S. Cl. 424/94.63; 435/69.1; 435/252.3; 435/471; 536/23.2; 435/212

(57) ABSTRACT

This invention relates to the production of recombinant lysostaphin in a homogenous form through the use of recombinant DNA molecules that express homogenous lysostaphin and host cells transformed with these DNA molecules. This invention also relates to the production of truncated forms of lysostaphin. The resulting lysostaphin preparations can be administered to those at infected or risk for infection by staphylococcal bacteria.

### CROSS-REFERENCE TO RELATED APPLICATIONS

This application is based on and claims the benefit of U.S. Provisional Application S.N. 60/341,804, filed Dec. 21, 2001 (Attorney Docket No. 07787.6007). The entire disclosure of this provisional application is relied upon and incorporated by reference herein.

### INTRODUCTION

Lysostaphin is an antibacterial enzyme first identified in Staphylococcus simulans (formerly known as S. staphylolyticus) in 1964. Lysostaphin is an endopeptidase capable of specifically cleaving the cross-linking pentaglycine bridges in the cell walls of staphylococci (27). Expressed in a single polypeptide chain, lysostaphin has a molecular weight of approximately 27 KDa

Because the cell wall bridges of Staphylococcus aureus contain a high proportion of glycine, lysostaphin is particularly effective in lysing S. aureus, a coagulase positive staphylococcus (27). Initial studies with lysostaphin also demonstrated that this enzyme could lyse Staphylococcus epidermidis, a coagulase negative staphylococcus. Subsequent studies found, however, that in comparison to S. aureus, lysing S. epidermidis required either higher concentrations of enzyme or longer incubation times depending on the strain of S. epidermidis (27). This strain-specific sensitivity to lysostaphin in S. epidermidis is thought to be due to differing amounts of glycine in the cell walls of each strain. Those strains that are more resistant to lysostaphin contain a higher proportion of serine in the cell wall rather than glycine (27).

Staphylococcal infections, such as those caused by S. aureus, are a significant cause of morbidity and mortality, particularly in settings such as hospitals, schools, and infirmaries. Patients particularly at risk include infants, the elderly, the immunocompromised, the immunosuppressed, and those with chronic conditions requiring frequent hospital stays. Further, the advent of multiple drug resistant strains of Staphylococcus aureus increases the concern and need for timely blocking and treatment of such infections. Indeed, the recent World Health Organization report entitled “Overcoming Astionicro Oral Resistance” detailed its concern that increasing levels of drug resistance are threatening to erode the medical advances of the recent decades. Among the issues raised are infections in hospitalized patients. In the United States alone, some 14,000 people are infected and die each year as a result of drug-resistant microbes acquired in hospitals. Around the world, as many as 60% of hospital-acquired infections are caused by drug-resistant microbes.

Patients at greatest risk are those undergoing inpatient or outpatient surgery, in the Intensive Care Unit (ICU), on continuous hemodialysis, with HIV infection, with AIDS, burn victims, people with diminished natural immunity from treatments or disease, chronically ill or debilitated patients, geriatric populations, infants with immature immune systems, and people with intravascular devices (2, 3, 4, 7, 8, 9, 13, 25, 26).

In infections caused by S. aureus, it appears that a principal ecological niche for S. aureus is the human anterior nares. Nasal carriage of staphylococci plays a key role in the epidemiology and pathogenesis of infection (2, 3, 7, 13, 23, 24, 25, 26). In healthy subjects, three patterns of S. aureus nasal carriage can be distinguished over time: approximately 20% of people are persistent carriers, approximately 60% are intermittent carriers, and approximately 20% apparently never carry S. aureus (7).

Chang et al. (1) studied 84 patients with cirrhosis admitted to a liver transplant unit. Overall, 39 (46%) were nasal carriers of S. aureus and 23% of these patients subsequently developed S. aureus infections as compared to only 4% of the non-carriers. A study of HIV patients (13) showed that 49% (114 of 296) of patients had at least one positive nasalculture for S. aureus. Thirty four percent of 201 patients were considered nasal carriers, with 38% of these being persistent carriers, and 62% intermittent carriers. Twenty-one episodes of S. aureus infection occurred in thirteen of these patients. Molecular strain typing indicated that, for six of seven infected patients, the strain of S. aureus isolated from the infected site was the same as that previously cultured from the nares, underlining the need for blocking of even apparently benign nasal colonization. The nasal S. aureus carrier patients were significantly more likely to develop S. aureus infection (P=0.04; odds ratio, 3.6; attributable risk, 0.44). This finding led the authors to conclude that nasal carriage is an important risk factor for S. aureus infection in HIV patients (13).

In a related patent application, Methods and Formulations for Eradicating or Alleviating Staphylococcal Nasal Colonization Using Lysostaphin, submitted concurrently herewith and specifically incorporated by reference, lysostaphin was shown to be effective in blocking and alleviating colonization of the nose by S. aureus when applied directly to the anterior nares in a viscous formulation. This lysostaphin formulation was effective against nasal colonization when applied concurrently with S. aureus in a cotton rat model or after nasal colonization was established in this animal model.

Lysostaphin may also provide therapy against active S. aureus infections of the skin, blood, and solid tissues. Among these are treatments for endocarditis (28) and systemic S. aureus (29) infections.

Given that ( ) S. aureus infections are prevalent in the general population; (2) S. aureus strains resistant to current antibiotics are emerging in the general population; (3) lysostaphin is very active against S. aureus including mutli-drug resistant strains and (4) a lysostaphin viscous formulation is effective in blocking and alleviating colonization in the nose (a major reservoir for S. aureus infection), there is a need in the art for a means of producing large quantities of lysostaphin for use in blocking and alleviating staphylococcal infections and colonization.

### BRIEF DESCRIPTION OF THE INVENTION

This invention relates to 1) recombinant truncated lysostaphin and 2) methods for the production of lysostaphin in a homogenous form. The lysostaphin of the invention is for use in patients at risk for staphylococcal infection. Populations at risk include infants with immature immune systems, patients admitted to the hospital for in-patient or out-patient surgical procedures, patients suffering from various conditions that predispose them to staphylococcal colonization and/or infections, or any patient prior to release from a hospital. The use of lysostaphin intranasally as a pre-release treatment will serve to reduce individual infections as well as to inhibit community spread of hospital-acquired staphylococcal strains.

The lysostaphins of the invention may be administered by application to skin, wounds, eyes, ears, lungs, mucus membranes of the nasal or gastrointestinal tracts. In one embodiment, the lysostaphins are administered by inhalation or other instillation into the nares or anterior nares of a patient.

Lysostaphin may be administered to a patient in several forms. In one embodiment, lysostaphin may be added to a viscous cream or liquid formulation. Viscous creams or liquids are acceptable for for intranasal administration and administration by other routes. Other possible carriers comprise natural polymers, semi-synthetic polymers, synthetic polymers, liposomes, and semi-solid dosage forms (10, 11, 12, 14, 16, 19, 21, 22). Natural polymers include, for example, proteins and polysaccharides. Semi-synthetic polymers are modified natural polymers such as chitosan, which is the deacetylated form of the natural polysaccharide, chitin. Synthetic polymers include, for example, dedrimers, polyphosphoesters, polyethylene glycol, poly (lactic acid), polystyrene sulfonate, and poly (lactide coglycolide). Semi-solid dosage forms include, for example, creams, ointments, gels, and lotions.

The invention includes compositions comprised of recombinant truncated lysostaphin. The invention also includes recombinant DNA molecules that encode one homogenous form of lysostaphin, which may or may not be truncated, host cells transformed with these DNA molecules, and methods of producing lysostaphin from these transformed cells. In one embodiment, lysostaphin expressed from the recombinant DNA molecule accumulates inside the transformed host cell in its final form. In another embodiment, lysostaphin expressed from the recombinant DNA molecule is secreted into the host cell media in its final form. Thus, the lysostaphin molecules produced by the methods of the invention do not require proteolysis to reach their final state. The methods of the invention may also be applied to any truncated or mutated functional form of lysostaphin to achieve expression and production of a homogenous population of lysostaphin molecules.

### BRIEF DESCRIPTION OF THE DRAWINGS

FIG. 1 provides a diagram summarizing the method of overlap extension PCR cloning.

FIG. 2 provides a restriction map of plasmid pJSB28, the amino acid sequence of the truncated lysostaphin protein encoded by pJSB28, and the nucleotide sequence encoding the truncated lysostaphin protein in pJSB28.

FIG. 3 provides a restriction map of plasmid pJSB20, the amino acid sequence of the truncated lysostaphin protein and signal polypeptide encoded by pJSB20, and the nucleotide sequence encoding the truncated lysostaphin protein and signal polypeptide in pJSB20. Underlined amino acids indicate the signal polypeptide sequence.

FIG. 4 demonstrates via an ELISA assay that host cells transformed with pJSB28 or pJSB20 produce intracellular and extracellular truncated lysosiaphin, respectively.

FIG. 5 demonstrates via a S. aureus lysing assay that host cells transformed with pJSB28 or pJSB20 produce functional truncated lysostaphin.

FIGS. 6A and 6B demonstrate via a colorimetric assay that the truncated form of lysostaphin is more active than the mature full length form.

FIGS. 7A-7D respectively provide restriction maps of the plasmids pRN5550, pNZ8148Scal, pLSS1C, and pLss12F. pLss12F encodes a truncated form of lysostaphin that has been bulk purified using methods disclosed in the instant invention.

FIG. 8 provides the nucleic acid sequence encoding truncated lysostaphin found in the plasmid pLss12f. The nucleic acid sequence contains a silent T to C mutation at position 255.

FIG. 9 shows the truncated form of lyphostatin on an SDS-PAGE reducing gel following the bulk purification protocol.

FIGS. 10-12 compare the bacteriocidal activity of homogeneous truncated and homogeneous mature lyphostaphins over a range of lysostaphin concentrations.

FIGS. 13 and 14 compare the bacteriocidal activity of homogeneous truncated and homogeneous mature lyphostaphins over a 60 minute time course in cultured human blood.

FIGS. 15 and 16 compare the bacteriocidal activity of homogeneous truncated and homogeneous mature lyphostaphins in an OD-drop assay.

### DETAILED DESCRIPTION OF THE INVENTION

Lysostaphin is naturally produced by bacteria as a pro-enzyme that is cleaved to produce the mature form of lysostaphin. The pro-enzyme contains a signal sequence for secretion of the enzyme, a tandem, repetitive motif typically consisting of 7 repeats of 13 amino acids, and the final full length (mature) protein of 246 amino acids. The enzyme that cleaves pro- lysostaphin is inconsistent in where it cleaves the molecule. But a lysostaphin molecule that has been incorrectly cleaved may be cleaved again by the same enzyme until the final form, beginning with amino acids “AATHE,” is produced (SEQ ID NO: 17). Accordingly, those in the art have agreed that mature, full length lysostaphin begins at the amino acid directly following the pro-sequence that is normally cleaved. Thus, mature lysostaphin begins with the amino acid sequence, “AATHE . . . ” When lysostaphin is isolated from bacteria, a snap shot of all the forms of lysostaphin present in the bacteria at the time of isolation results. Several forms of lysostaphin are present in the resulting preparation: 1) less active pro-lysostaphin; 2) active mature lysostaphin (“AATHE” form); and 3) intermediate forms of lysostaphin, i.e., molecules at various stages of maturation that have different amounts of pro-sequence not yet cleaved from the molecule. Thus, preparations from natural sources result in a heterologous population of active and less active lysostaphins that begin at different amino acids.

The presence of less active forms of lysostaphin dilutes out the concentration of fully active lysostaphin in the preparation, thus decreasing the specific activity of a composition containing naturally derived lysostaphin. In contrast, recombinant lysostaphin preparations contain only a fully active, mature or truncated form of lysostaphin. In such preparations, there is no less active form to dilute out the activity of the recombinant molecules. Thus, the specific activity of a composition made with recombinant lysostaphin will be higher than one made with naturally-derived lysostaphin.

Under current production methodologies, the lysostaphin is expressed in B. sphaericus, from a genetically engineered plasmid, as a pro-protein that is post-translationally processed to its full length active form. The active enzyme is then isolated from the growth medium. As with natural forms of lysostaphin, this product, however, consists of a mixture of polypeptides ranging from 248 to 244 amino acids in length due to differential proteolysis of the pro-enzyme (5). As such, lysostaphin sequences, for example those in Ambicin L (Ambi, Purchase NY), may begin with the sequences “RAATHE . . . , ” “LAATHE . . . ,” “AATHE . . . ,” or “THE . . . .” (SEQ ID NOS: 15-17; SEQ ID NO: 13).

One aspect of the invention relates to compositions comprising homogenous recombinant truncated lysostaphin. In one aspect of this embodiment, the gene for lysostaphin was genetically truncated to remove the lysostaphin signal sequence, the repetitive elements (the “pro” domain), and the first two alanine amino acids in the full length lysostaphin amino acid sequence. This truncated lysostaphin sequence, beginning with the amino acids “THE,” was fused to either an initiating methionine for intracellular expression or a signal sequence allowing the secretion of a single species of truncated lysostaphin into the periplasmic space of E. coli. In removing the first two amino acids of the mature lysostaphin sequence (ala-ala), the inventors have generated a form of lysostaphin with improved antistapylococcal activity as compared to the mature full length protein. Other embodiments may include other truncations.

In other aspects of the invention, homogenous truncated lysostaphin may be used in a pharmaceutical composition for treatment of staphylococcal nasal colonization or as a therapy for various active S. aureus infections. Government standards often require that an ingredient added to a pharmaceutical composition must be a homogenous form. A mixture of different chemical forms of the ingredient is less likely to be incorporated into a pharmaceutical composition. Thus, the homogenous, truncated lysostaphin of the invention is well-suited for use in pharmaceutical compositions.

The present invention pharmaceutical compositions comprises a therapeutically effective amount of a lysphostatin of the invention, together with a pharmaceutically acceptable carrier. Pharmaceutically acceptable carriers can be sterile liquids, such as water, oils, including petroleum oil, animal oil, vegetable oil, peanut oil, soybean oil, mineral oil, sesame oil, and the like. Saline solutions, aqueous dextrose, and glycerol solutions can also be employed as liquid carriers, particularly for injectable solutions. Suitable pharmaceutical carriers are described in Remington's Pharmaceutical Sciences, 18th Edition (13), which is herein incorporated by reference.

A therapeutically effective amount is an amount reasonably believed to provide some measure of relief, assistance, prophylaxis, or preventative effect in the treatment of the infection. A therapeutically effective amount may be an amount believed to be sufficient to block a bacterial colonization or infection. Similarly, a therapeutically effective amount may be an amount believed to be sufficient to alleviate an existing bacterial infection.

Another aspect of the invention is directed to methods for the production of homogenous lysostaphin. Unlike the current production methods discussed above, the instant invention is designed to express lysostaphin proteins all of which begin at the same amino acid by removing the need for proteolysis after expression of the recombinant protein. The method of the invention uses DNA molecules, discussed below, that express lysostaphin in its final form, whether full length or truncated. Because the resulting lysostaphin molecules do not require unpredictable proteolysis to reach their final form, the instant invention results in a substantially homogenous preparation of lysostaphin with each molecule beginning at the same amino acid. The homogenous lysostaphin of the invention also carries with it the added advantage of simplifying detection of contaminants. Specifically, contaminants from the bacterial cells expressing lysostaphin may remain in the preparation. Finding or eliminating those contaminants in a preparation is more difficult when several forms of lysostaphin are present in the preparation, given the required chemical analysis to test for purity. Thus, a system that expresses homogenous lysostaphin is more readily adaptable to producing pure lysostaphin, free of contaminants, for potential clinical use.

Another aspect of the invention is directed to a recombinant DNA molecule encoding a homogenous form of lysostaphin. In one embodiment, the recombinant DNA molecule may be a plasmid that is comprised of an origin of replication, a gene conferring antibiotic resistance to the transformed host cell, a promoter region operatively linked to a DNA sequence encoding lysostaphin, a signal sequence, and a termination sequence. The recombinant DNA molecule may also be in an alternate form such as a cosmid or linearized DNA. The recombinant DNA may also be incorporated into the host cell genome for purposes of stability during commercial, pharmaceutical production. In another embodiment, the recombinant DNA molecule may encode full length lysostaphin, truncated lysostaphin, or a lysostaphin variant. The recombinant DNA molecule of the invention may encode lysostaphin that localizes inside the cell (i.e., intracellular expression) or may encode lysostaphin with a signal for secretion of lysostaphin into the host cell media (i.e., extracellular expression).

The recombinant DNA molecule of the invention also includes DNA molecules that do not express lysostaphin protein, but that contain a nucleotide sequence that is capable of expressing lysostaphin protein if operably linked to appropriate genetic control elements (i.e., a promoter).

The term “lysostaphin,” as used herein, means full length lysostaphin, any lysostaphin mutant or variant, any lysostaphin truncation, any recombinantly expressed lysostaphin protein, or a related enzyme that retains the proteolytic ability, in vitro and in vivo, of proteolytic attack against glycine-containing bridges in the cell wall peptidoglycan of staphylococci. Modified full-length lysostaphin or lysostaphin variants may be generated by post-translational processing of the protein (either by enzymes present in a host cell strain or by means of enzymes or reagents introduced at any stage of the process) or by mutation of the structural gene.

Mutations may include deletion, insertion, domain removal, point and replacement mutations. Lysostaphin includes, for example, lysostaphin purified from S. simulans, Ambicin L (Nutrition 21, Inc.), purified from B. sphaericus, or lysostaphin purified from a recombinant expression system such as the instant invention. Truncated lysostaphin includes any lysostaphin protein in which one or more amino acids have been removed from the protein's amino terminus, carboxy terminus, or both. Lysostaphin variants may also be expressed in a truncated form.

The term “express,” as used herein, refers to the process by which messenger RNA is transcribed from a DNA template such that the messenger RNA is then translated into the amino acid sequence that forms a protein. Thus, a DNA molecule expresses lysostaphin when it contains nucleotide sequences that may be transcribed into messenger RNA that will be translated into a lysostaphin protein. For the purposes of this invention, the term “express” is essentially equivalent to the term “functionally encode.”

The term “origin of replication,” as used herein, means a DNA sequence that allows an extrachromosomal piece of DNA, such as a plasmid, to duplicate itself independently of chromosomal replication. The origin of replication often binds host cell proteins that participate in DNA replication in the cell.

The term “promoter,” as used herein, refers to a DNA sequence that facilitates the production of messenger RNA by the process of transcription. A promoter is “operatively linked” to a gene when the initiation of the transcription process at the promoter leads to the production of messenger RNA encoded by that gene.

The term “signal sequence,” as used herein, refers to a DNA sequence that encodes an amino acid sequence that signals the host cell to perform a specific task with the resulting protein. For example, a signal sequence may instruct a host cell to secrete the encoded protein rather than to keep it inside the cell. The term “termination sequence,” as used herein, means a DNA sequence that stops the process of transcription. A terminator sequence normally follows the DNA sequence of the gene of interest in a plasmid.

Another aspect involves transforming a host cell with the recombinant DNA encoding lysostaphin. The term “host cell,” as used herein means any cell, prokaryotic or eukaryotic, including animal and plant cells, that may be transformed or transfected with the recombinant DNA of the invention. In one embodiment of the invention, the host cell is a bacterium, for example, Eschericia. coli, Lactococcus lactis, Bacillus sphaericus, and related organisms. Genetic elements in the recombinant DNA of the invention, such as the origin of replication, the promoter, the signal sequence, and the termination sequence are often host cell specific. Thus additional embodiments include recombinant DNA molecules that contain elements for these functions that work in the specific host cell used.

The term “transform, ”as used herein, means the introduction of a DNA molecule into a bacterial cell. Bacterial cells are made “competent” when they will readily receive foreign DNA molecules. Methods for making bacterial cells competent and for transforming these competent cells are standard and known to those of skill in the art. Bacteria may also be transformed by electroporation. The term “transfect,” as used herein, means the introduction of a DNA molecule into a mammalian host cell. A mammalian host cell may be transfected by several common methods including electroporation, calcium phosphate precipitation, DEAE-Dextran, and liposome reagents (i.e., Lipofectin).

Another aspect of the invention relates to the production of active homogenous lysostaphin and active truncated homogenous lysostaphin from host cells that are transformed or transfected with a recombinant DNA molecule expressing lysostaphin. The term “homogenous lysostaphin,” as used herein, means a preparation of lysostaphin in which all of the lysostaphin molecules begin at the same amino acid in the lysostaphin protein sequence. Recombinant lysostaphin produced in bacteria may have residual, uncleaved F-Met on the N-termini of some molecules and recent studies show that 20% and 2% of recombinant molecules produced in in L. lactis and E. coli, respectively, contain residual F-Met moieties. Consequently, a recombinant homogeneous lyphostatin may include bacterially-produced lysostaphin or truncated lysostaphin with and without N-terminal F-Met residues. In some embodiments, greater than 50%, 60%, 70%, 80%, or 90% of the recombinant molecules do not have residual F-Met moieties.

The term “heterogenous lysostaphin,”—0 as used herein, means a preparation of lysostaphin that contains a mixture of lysostaphin molecules that begin at different amino acids in the lysostaphin sequence. The active homogenous lysostaphin may be either full length lysostaphin or truncated lysostaphin. A lysostaphin protein is “active” when it exhibits proteolytic activity, in vitro and in vivo, to cleave the specific cross-linking polyglycine bridges in the cell walls of staphylococci. A lysostaphin protein has “enhanced staphylolytic activity” when it exhibits increased proteolytic activity against polyglycine bridges in the cell walls for staphylococci as compared to full length lysostaphin. When lysostaphin is expressed inside the host cell, the host cell may be lysed and lysostaphin isolated from the resulting cell extract. When lysostaphin is secreted into the host cell media, lysostaphin may be concentrated and then purified using standard techniques such as column chromatography, hydrophobic purification, and ion-exchange chromatography. For purposes of large scale production, host cells may be grown in, for example, large roller bottles or large volume fermenters.

Homogenous, recombinant lysostaphin may be added to a variety of carriers. Such vehicles increase the half-life of the lysostaphin in the nares following instillation into the nares. These carriers comprise natural polymers, semi-synthetic polymers, synthetic polymers, liposomes, and semi-solid solid dosage forms (10, 11, 12, 14, 15, 18, 20, 21). Natural polymers include, for example, proteins and polysaccharides. Semi-synthetic polymers are modified natural polymers such as chitosan, which is the deacetylated form of the natural polysaccharide, chitin. Synthetic polymers include, for example, dedrimers, polyphosphoesters, polyethylene glycol, poly (lactic acid), polystyrene sulfonate, and poly (lactide coglycolide). Semi-solid dosage forms include, for example, creams, ointments, gels, and lotions. These carriers can also be used to microencapsulate lysostaphin molecules or can be covalently linked to lysostaphin.

The resulting lysostaphin compounds may be administered to a patient at risk for staphylococcal infection by several routes. Patients at risk, include health care workers, newborns and premature infants, persons undergoing inpatient or outpatient surgery, burn victims, patients receiving indwelling catheters, stents, joint replacements and the like, geriatric patients, and those with genetically, chemically or virally suppressed immune systems. Among non-human patients, those at risk include zoo animals, herd animals, and animals maintained in close quarters.

Representative patients include any mammal subject to S. aureus, or other staphylococcal infection or carriage, including humans and non-human animals such as mice, rats, rabbits, dogs, cats, pigs, sheep, goats, horses, primates, ruminants including beef and milk cattle, buffalo, camels, as well as fur-bearing animals, herd animals, laboratory, zoo, and farm animals, kenneled and stabled animals, domestic pets, and veterinary animals.

In one embodiment, a lysostaphin composition may be instilled into the nares of a patient. In alternate embodiments, a lysostaphin composition may be rubbed onto the skin, applied to an open wound, or injected for systemic administration. In addition, these lysostaphin compositions may be administered in conjunction with antibiotic anti-staphylococcal drugs including antibiotics like mupirocin and bacitracin; anti-staphylococcal agents including lysozyme, mutanolysin, and cellozyl muramidase; anti-bacterial peptides like nisin; and lantibiotics, or any other lanthione -containing molecule, such as subtilin. The compositions of the invention may also include anti-staphylococcal agents including anti-staphylococcal monoclonal antibodies, for example antibodies directed against peptidoglycan (PepG), described in U.S. Provisional Application 60/343,444, filed Dec. 21, 2001, and Multifunctional Monoclonal Antibodies Directed To Peptidoglycan of Gram-Positive Bacteria, filed herewith, both of which are incorporated by reference.

The recombinant lysostaphins and pharmaceutical compositions of the invention may be administered by intravenous, intraperitoneal, intracorporeal injection, intra-articular, intraventricular, intrathecal, intramuscular or subcutaneous injection, or intranasally, dermally, intradermally, intravaginally, orally, or by any other effective method of administration. The composition may also be given locally, such as by injection to the particular area infected, either intramuscularly or subcutaneously. Administration can comprise administering a pharmaceutical composition by swabbing, immersing, soaking, or wiping directly to a patient. The treatment can also be applied to objects to be placed within a patient, such as dwelling catheters, cardiac valves, cerebrospinal fluid shunts, joint prostheses, other implants into the body, or any other objects, instruments, or appliances at risk of becoming infected with a staphylococcal bacteria, or at risk of introducing such an infection into a patient.

The present invention is further illustrated by the following examples that teach those of ordinary skill in the art how to practice the invention. The following examples are merely illustrative of the invention and disclose various beneficial properties of certain embodiments of the invention. The following examples should not be construed as limiting the invention as claimed.

### EXAMPLES

### Example 1

### Construction of an Expression Plasmid for Intracellular Expression of Homogenous Truncated Lysostaphin

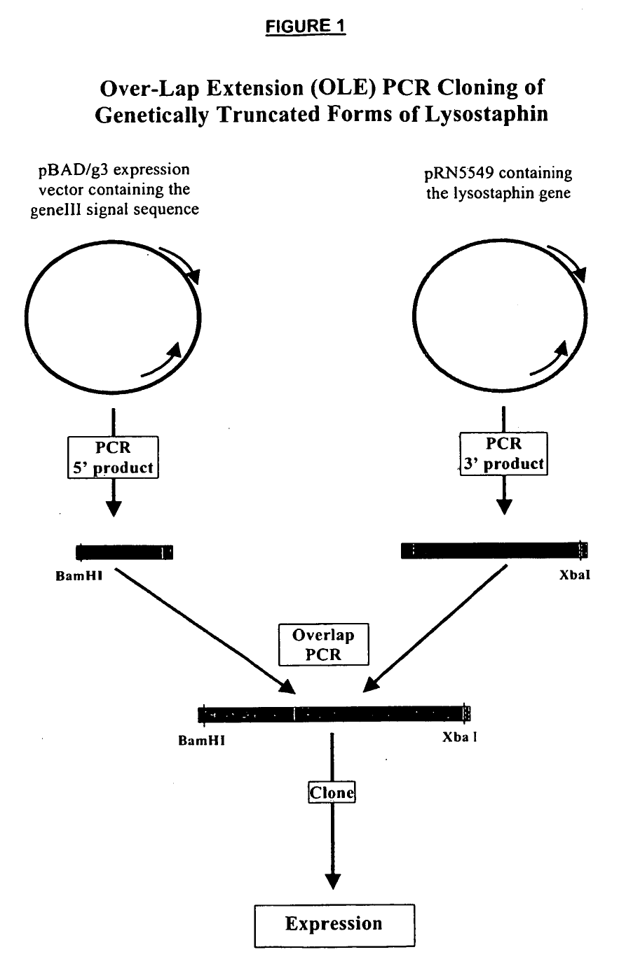

Polymerase Chain Reaction (PCR) was used to amplify a fragment of the lysostaphin gene and the resulting fragment was cloned into pBAD/glll expression vector (Invitrogen). A form of truncated lysostaphin was generated and fused directly to a translation start methionine for intracellular expression in E. coli. FIG. 1 provides the cloning strategies used for production of the plasmid of this example and Example 2 below.

To complete the fusion of the lysostaphin expression cassettes, the 5′ and 3′ ends were amplified from different templates and then connected using overlap extension PCR (“OLE-PCR”). Specifically, the 5′ fragment containing the upstream expression components was amplified from the pBAD/gill plasmid and the 3′ end, containing the lysostaphin coding sequence, was amplified from a sample of host cells containing the lysostaphin gene, provided by Ambi, Inc. The PCR amplification reactions for the 5′ fragment contained 10 ng of template DNA, 20 pmoles of primers JSBX-29 and JSBX-53 (see Table 1), 2.5 units of ExTaq polymerase (PanVera), 1x ExTaq reaction buffer, 200 μM dNTP, 2 mM MgCl₂ in a 50 μl reaction volume. The template was denatured by an initial incubation at 96° C. for 3 min. The products were amplified by 25 thermal cycles of 96° C. for 30 sec., 56° C. for 30 sec., 72° C. for 30 seconds. The PCR amplification reactions for the 3′ fragment contained 10 ng of template DNA, 20 pmoles of primers JSBX-34 and JSBX-52 (see Table 1), 2.5 units of ExTaq polymerase, 1x ExTaq reaction buffer, 200 μM dNTP, 2mM MgCl₂ in a 50 μl reaction volume. The template was denatured by an initial incubation at 96° C. for 3 min. The products were amplified by 25 thermal cycles 96° C. for 30 sec., 52° C. for 30 sec., 72° C. for 30 seconds. The PCR products from the successful reactions were purified using the Nucleospin PCR Purification system (Clontech) per the manufacturer's procedure.

TABLE 1

| | | SEQ |
|---|---|---|
| | | ID |
| Primer | Primer Sequence (5′ to 3′) | NO: |
| JSBX-29 | ATTAGCGGATCCTACCTGAC | 1 |
| JSBX-32 | CCATTGTGCTGAATGTTCATGTGTGCTATGGCTATAGAACGGC | 2 |
| JSBX-33 | GGTGCCGTTCTATAGCCATAGCACACATGAACATTCAGCACAATGG | 3 |
| JSBX-34 | TTATTCTTCTAGATCACTTTATAGTTCCCCAAAGAAC | 4 |
| JSBX-35 | CTATGCCATAGCATTTTTATCC | 5 |
| JSBX-36 | AGCCAAGCTGGAGACCG | 6 |
| JSBX-52 | GGGCTAACAGGAGGAAGCTTCCATGACACATGAACATTCAGCAC | 7 |
| JSBX-53 | TGTGTCATGGAAGCTTCCTCCTGTTAGCCC | 8 |

OLE-PCR was then performed using 2 μL of the purified 5′ fragment, 0.5 μL of the purified 3′ fragment, 20 pmol of primers JSBX-29 and JSBX-34, 2.5 units of ExTaq polymerase, 1x ExTaq reaction buffer, and 200 μM dNTP, 2 mM MgCl₂ in a 50 μl reaction volume. The template was denatured by an initial incubation at 96° C. for 3 min. The products were amplified by 30 thermal cycles 96° C. for 30 sec., 56° C. for 30 sec., 72° C. for 30 seconds. The first 5 cycles were performed without the addition of primer DNA to optimize the likelihood that the 5′ and 3′ sections, when denatured, would overlap appropriately to allow amplification of the entire lysostaphin sequence and accessory elements (i.e., signal sequence). The PCR products from successful reactions were purified using the Nucleospin PCR Purification system per manufacturer's instructions.

The PCR products, approximately 1000 base pairs in length, were then digested with restriction endonucleases BamHl and Xbal, and cloned into a bacterial vector, pBAD/gIII, for protein expression. The digested PCR fragments were ligated into de-phosphorylated, BamHl and Xbal digested pBAD/gIII, using ligase (Promega) and following the manufacturer's instructions using a 3:1 insert to vector molar ratio. One half (5 μl) of the ligation reactions were used to transform competent TOP10 cells (Invitrogen) per the manufacturer's instructions. Bacterial clones containing plasmids with DNA inserts were identified using diagnostic restriction enzyme digestions with PflMI (New England Biolabs). The expected pattern of banding in this diagnostic digest was one band at 4000 bp and one band at 800 bp for pJSB20 and 4000 bp and 750. bp for pJSB28. DNA sequencing was performed using cycle sequencing reactions primed by JSBX-35 (SEQ ID NO: 5) and JSBX-36 (SEQ ID NO: 6) and analyzed on a CEQ2000 capillary sequencer (Beckman/ Coulter). Sequencing was carried out by mixing 500 ng of plasmid preparation, 10 ng of sequencing primer, and 8 pi of CEQ2000 sequencing mix (Beckman Coulter PN 608000). The sequencing samples were incubated at 96° C. for 2 minutes and then amplified by 96° C. 20 seconds, 50° C. for 20 seconds, and 60° C. for 4 minutes for a total of 40 cycles.

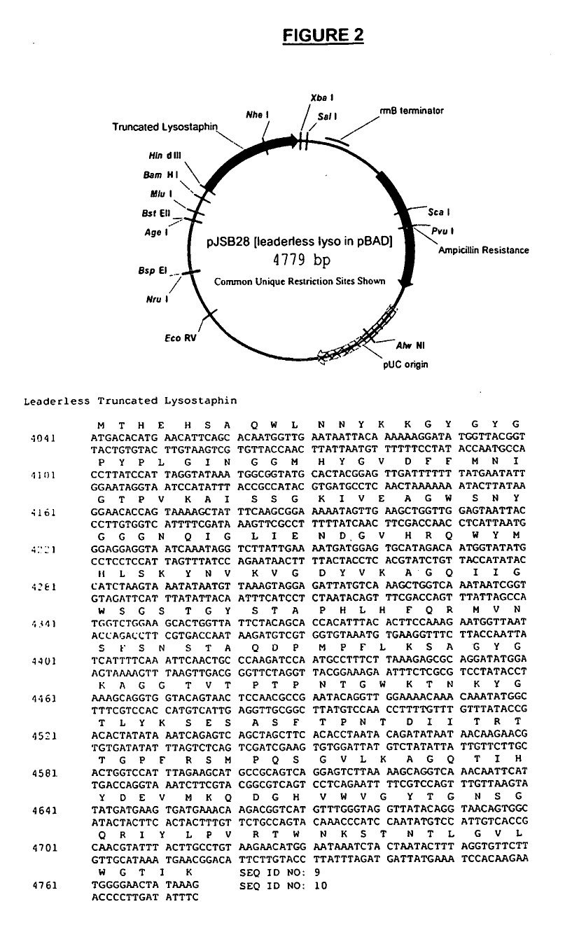

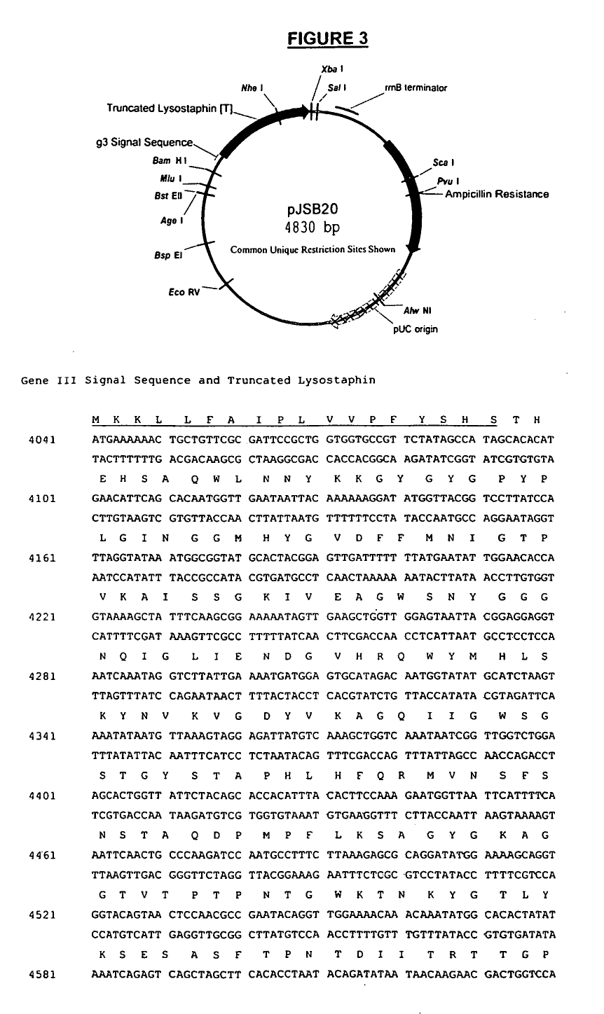

The resulting plasmid, pJSB28, is shown in FIG. 2. This figure also shows the sequence of the truncated lysostaphin gene that is present in this plasmid (SEQ ID NO: 9 for amino acid sequence-and SEQ ID NO: 10 for nucleotide sequence).

### Example 2

### Construction of an Expression Plasmid for Extracellular Expression of Truncated Lysostaphin

As described above, PCR was used to amplify a fragment of the lysostaphin gene and the resulting fragment was cloned into pBAD/gIII expression vector (Invitrogen). In this example, a form of truncated lysostaphin was generated and fused to a signal sequence for secretion into the periplasmic space of E. coli. OLE-PCR was also used to complete the fusion of the lysostaphin expression cassettes as described above, except that the 5′ fragment contained the upstream expression components and the signal sequence for extracellular expression. These 5′ components were also amplified from the pBAD/gIII plasmid. The PCR amplification reactions for the 5′ fragment were as described above except primers JSBX-29 and JSBX-32 were used. Likewise, the 3′ fragment was amplified as described above except that primers JSBX-34 and JSBX-33 were used. OLE-PCR, the cloning of the truncated lysostaphin insert into the pBAD/gIII vector, the diagnostic digest, and subsequent sequencing were all performed as described above.

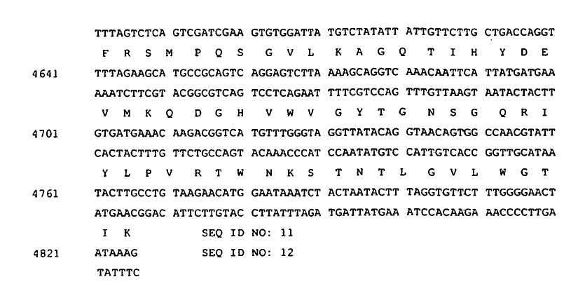

The resulting plasmid, pJSB20, is shown in FIG. 3. This figure also shows the sequence of the truncated lysostaphin gene and signal sequence that is present in this plasmid (SEQ ID NO: 11 for amino acid sequence and SEQ ID NO: 12 for nucleotide sequence).

### Example 3

### Small Scale Production of Active Truncated Lysostaphin in E.coli

Overnight cultures of TOP10 cells transformed with pJSB20 or pJSB28 grown at 37° C. were diluted 1:100 in 200 ml LB media supplemented with 100 μg/mL ampicillin. When the OD₆₀₀ reached 0.5, arabinose was added to 0.2% to induce expression of the lysostaphin protein. The cultures were then allowed to grow for 48 hours at 30° C. Cells were pelleted by centrifugation at 5000×g for 15 minutes. In the case of the intracellular lysostaphin (pJSB28), the cell pellet was treated with B-Per extraction reagent (Pierce) supplemented with 0.125M NaCl following the manufacturer's instructions and the supernatant was collected and analyzed for the presence of lysostaphin and whether the enzyme was active. In the case of secreted lysostaphin (pJSB20), the media supernatant was collected, concentrated using an Amicon spiral ultrafiltration concentrator (S1Y-10 with a 10 KD cutoff) and analyzed for the presence of lysostaphin and for lysostaphin activity.

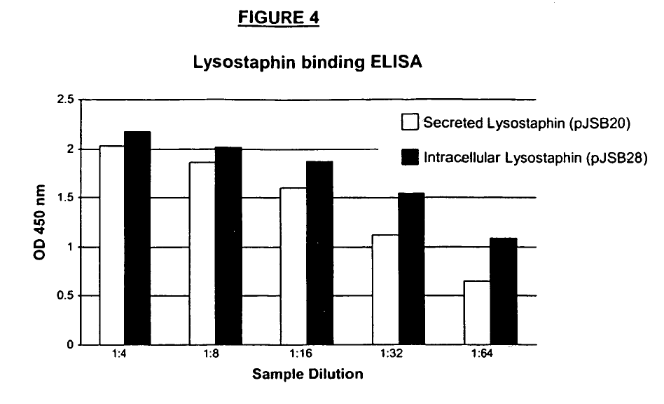

To determine if lysostaphin was expressed either intracellularly or by secretion into the host cell media, the presence of lysostaphin in the resulting sample was analyzed by ELISA. Polyclonal anti-lysostaphin was generated in rabbits by primary vaccination with 100 μg of lysostaphin to the popliteal lymph nodes. The animals were given 100 μg booster injections once per month for the following three months before being bled to produce the polyclonal serum. In the ELISA, 100 μl of the resulting rabbit polyclonal anti-lysostaphin serum was diluted 1:10,000 in PBS and used to coat wells of a 96-well microtiter plate (NUNC, Macrosorb) overnight at 4° C. The plates were then washed with PBS and blocked with 100 μl/well of 1% BSA in PBS at room temperature for 30-60 minutes. Experimental samples and the lysostaphin standard (Ambicin L, AMBI, Inc.) were diluted in PBS with 0.1% Tween and 0.1% BSA (PBS-T-BSA). The anti-lysostaphincoated, blocked plates were then washed with PBS-T four times. The samples and standard dilutions were then transferred (100 μl/well) onto an anti-lysostaphin coated plate and incubated for 30-60 minutes at room temperature. The plate was then washed 4 times with PBS-T. The coating polyclonal antibody was also used as the detection antibody by biotinylating the antibody with Biotin-sulfo-N-hydroxysuccinimide caprylate (Bioaffinity Systems, Inc.) according to the manufacturer's instructions. This biotinylated antibody preparation was then diluted 1:800 in PBS-T-BSA and added at 100 μL/well. The plate was incubated 30-60 minutes at room temperature and then washed 4 times with PBS-T. ExtraAvidin-HRP (Sigma Cat# E2886) was diluted 1:8000 in PBS-T-BSA and then 100 μL/well was added to the plate which was incubated for 30-60 minutes. The plate was washed-4 times with PBS-T. One hundred microliters per well of TMB-Microwell Substrate (BioFx Cat# TMBW 01000-01) was added and the reaction was allowed to proceed for 3-5 minutes before being stopped by the addition of TMB Stop reagent (BioFx Cat# STPR 0100-01). Absorbance was then read at 450 nm. As shown in FIG. 4, both the pJSB28 and pJSB20 DNA plasmids expressed lysostaphin when transformed into E. coli host cells.

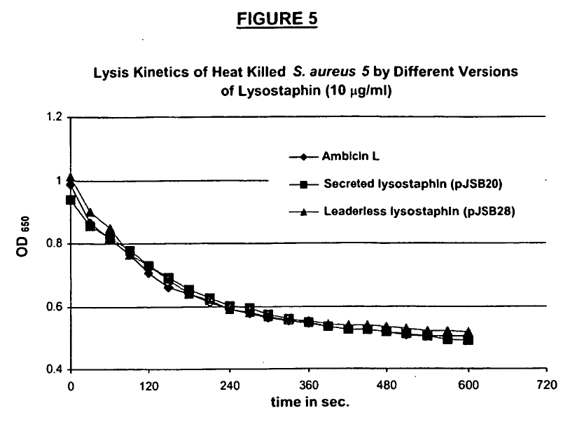

To determine if the lysostaphin expressed by pJSB28 and pJSB20 was active, a lysis assay was used. In this assay, S. aureus bacteria were heat killed and then incubated with varying concentrations of the lysostaphin preparation. Lysostaphin activity was detected when the experimental preparation caused the bacterial suspension to lose its turbidity and become clear. Specifically, the lysostaphin positive control (Ambicin L) and samples were diluted to concentrations of 200, 100, 50 and 10 μg/ml in 50 μl of PBS. S. aureus 5 was heat killed by incubation at 56° C. for 3 hours. These heat killed bacteria were suspended to approximately OD₆₅₀0.9-1.0 in PBS. Then, 450 μl of this suspension was placed in a disposable cuvette and the OD₆₅₀ was determined. This was the 0 time point. Fifty microliters of the sample or control was then added, bringing the final concentration of lysostaphin in the samples to 20, 10, 5 and 1 μg/ml. The OD₆₅₀ of the suspension was thereafter measured every 60 seconds for 10 minutes. FIG. 5 is a typical kinetic curve for the 10 μg/mL sample of lysostaphin expressed by pSJB28 and pSJB20. PBS alone was used as a negative control in the assay. In this assay, the OD₆₅₀ never reaches zero due to the presence of bacterial debris that remains even after complete lysis of the sample.

### Example 4

### Truncated LVsostaphin has Enhanced Staphylolytic Activity Over Full Length Lysostaphin

The staphylolytic activity of the truncated “THE” form of lysostaphin was compared to the staphylolytic activity of the full length “AATHE” form of lysostaphin using a fluorescence emission assay as described in Kline et al. (6). In the current assay, fluorescamine was substituted for TNBS to detect amines revealed when lysostaphin cuts the N-acetyl hexaglycine substrate.

Lysostaphin samples were prepared by first determining the optical density of the enzyme using a Spectrophotometer at OD280 nm (OD₂₈₀). The spectrophotometer was zeroed with lysostaphin dilution buffer. 1:10 dilutions were prepared by adding 100 μl of enzyme solution to 900 μl lysostaphin dilution buffer (20 mM Sodium Acetate, and 0.5% Tween 20, pH 4.5) and vortex mixing. The samples were then read at OD_{280.} The concentration (mg/ml) of lysostaphin in the enzyme sample was calculated by multiplying the resulting OD₂₈₀ values by the dilution factor of 10 and by the extinction coefficient of 0.41. For use in this assay, each lysostaphin sample was then diluted to 40 μg/ml, using lysostaphin dilution buffer.

The hexaglycine substrate was prepared by as described by Kline et al. (6). Specifically, a 10 mM stock was prepared by resuspending 40.2 mg N-acetylhexaglycine (N-Ac-Hex; FW=402) in 1 ml of water, adding sodium hydroxide until the solid dissolved in solution. The final volume of the N-Ac-Hex stock was adjusted to 10 mls by adding substrate/assay buffer (5 mM Trisodium citrate, 1 mM Disodium EDTA, 0.1 M Sodium Borate, pH 8.0). An aliquot of this stock was further diluted 1:10 with substrate/assay buffer before use in the assay.

Each lysostaphin sample was transferred to a microwell plate and diluted 1:3 (starting from 40 μg/ml) in dilution buffer. After dilutions were complete, the final volume in each well was 60 μl. Blank wells lacking each component were also prepared. Each sample was done in triplicate. Once the lysostaphin sample dilutions were prepared, 60 μl of N—Ac-Hex substrate was added to each well.

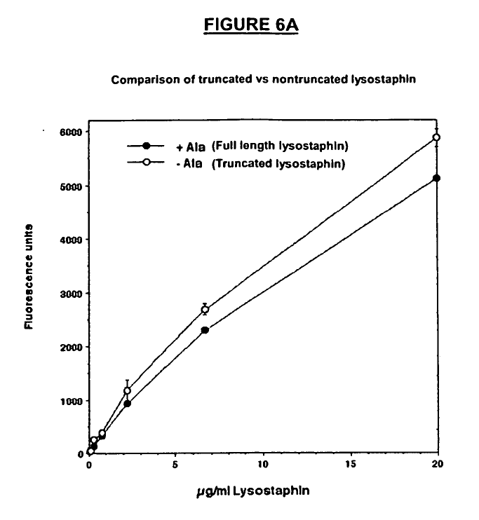

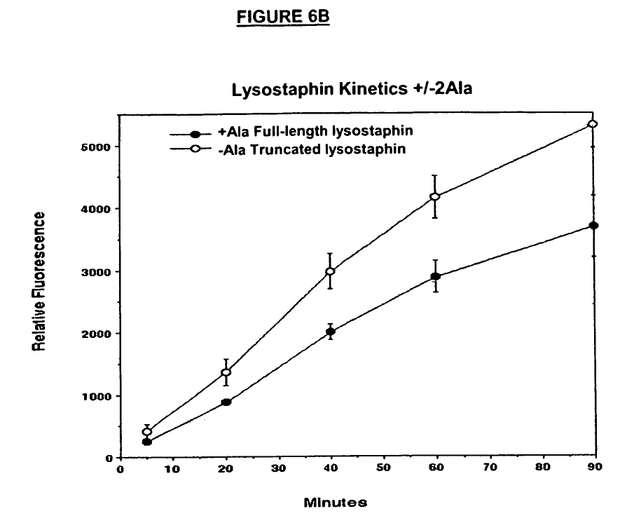

The assay plates were then incubated at 37° C. for 1 hour. 40 μl of 0.3 mg/ml fluorescamine (Molecular Probes) in acetone was then added to each well and the sample read immediately. Samples were read using a fluorescent plate reader (Molecular Devices) which was set at 390 nm excitation, 475 nm emittence, and 420 nm cut off. Negative controls consisted of the matching wells) containing lysostaphin, buffers, and fluorescamine, but lacking N—Ac-hexaglycine. The fluorescence of wells containing the corresponding concentration of lysostaphin but lacking the N-Ac-Hex substrate were subtracted from each reading. Samples were done in triplicate, the values averaged, and the standard deviation calculated. The results of this experiment are shown in FIG. 6A.

A time course experiment was also performed using a uniform concentration of lysostaphin in each experimental well. As described above, negative controls consisted of the matching well(s) containing lysostaphin, buffers, and fluorescamine, but lacking N—Ac-hexaglycine. Experimental wells contained lysostaphin at a final concentration of 20 μg/ml. Samples were incubated at 37° C. for 5 minutes, 20 minutes, 40 minutes, 60 minutes, and 90 minutes. When the incubation period lapsed, 40 μl of 0.3 mg/ml fluorescamine in acetone was added to each well and the sample read immediately. Samples were read and analyzed as described above. The results of this experiment are shown in FIG. 6B.

FIGS. 6A and 6B demonstrate that the truncated “THE” form of lysostaphin was more active than the full length “AATHE” form. The colorimetric assay monitors the action of each lysostaphin molecule on a substrate molecule whereas the S. aureus viability assay simply measures whether there was enough lysostaphin activity to kill a S. aureus bacterium. FIGS. 6A and 6B show that truncated lysostaphin has increased staphylolytic activity over the full length “AATHE” form. In turn, lysostaphin compositions produced with this truncated lysostaphin would provide enhanced staphylolytic activity over compositions made with heterologous lysostaphin or homologous full length lysostaphin.

### Example 5

Large scale purification of Active Truncated Lystostaphin in L. lactis Cloning of the lysostaphin gene:

The entire lysostaphin gene was amplified from the plasmid pRN5550 using the polymerase chain reaction as generally described above (PCR). pRN5550 had previously been used for lystostaphin production at AMBI Inc., Purchase, NY. The resulting amplified PCR product contained the coding region for the production of lysostaphin, a 5′ TTG codon for translation initiation, and a 3′ Xbal restriction site for cloning purposes. This PCR product was then digested with Xbal and ligated into a standard L. lactis expression plasmid, pNZ8148Scal, which had been cut with Scal and Xbal. This ligation created the expression plasmid pLss1C.

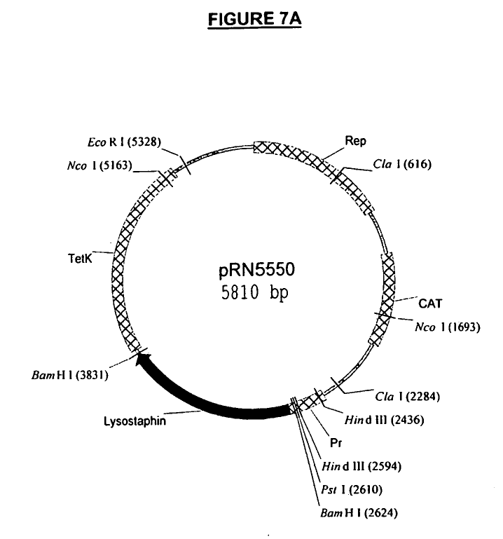

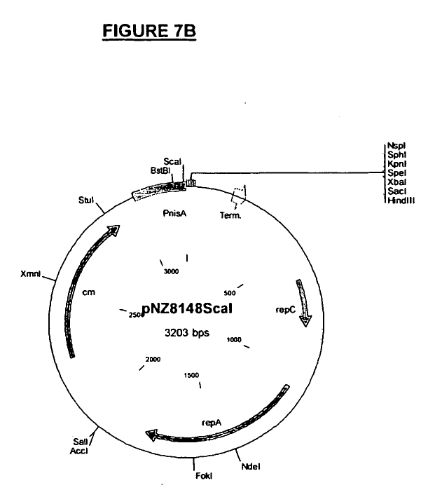

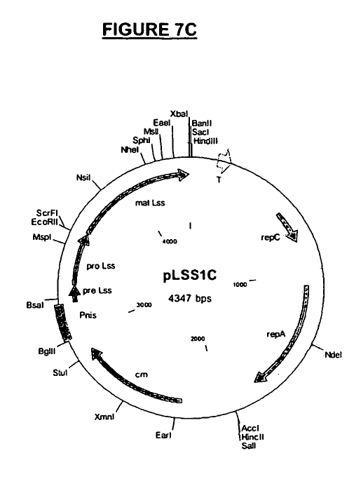

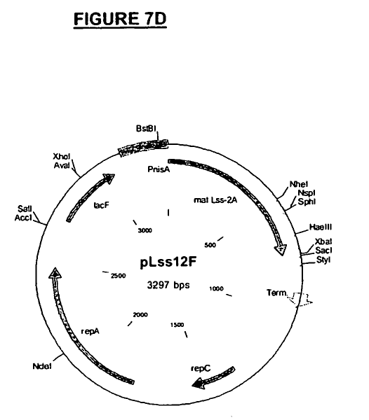

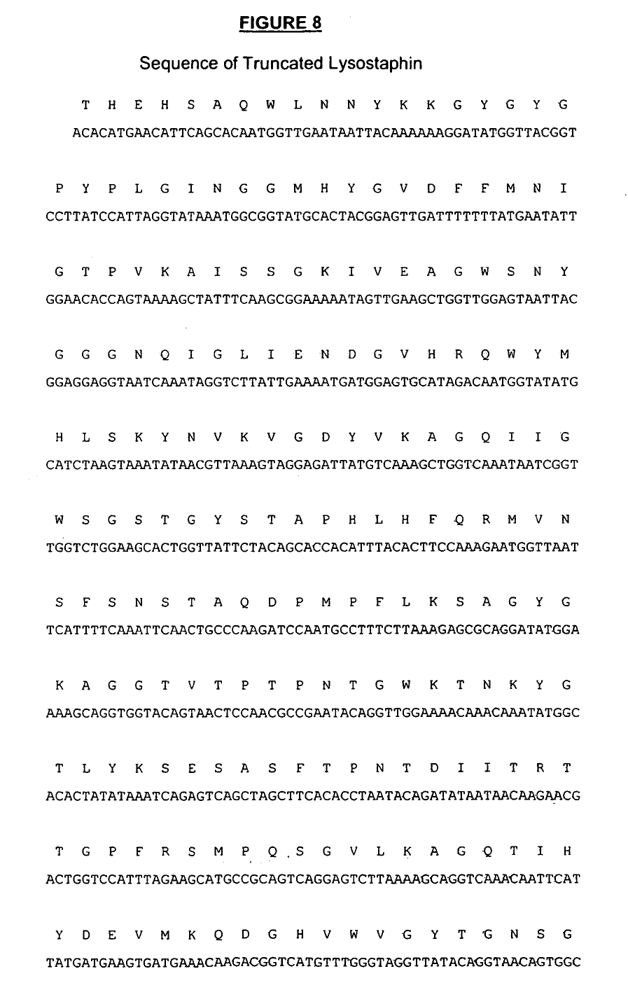

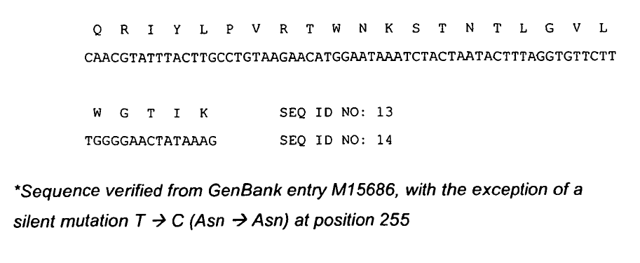

Sequence analysis confirmed that the lysostaphin coding sequence in pLss1C was correct. This included a known silent mutation at position 256 (T to C; Asn to Asn) present in the lystostaphin coding sequence in pRN5550. (SEQ ID NO: 13 for amino acid sequence and SEQ ID NO: 14 for nucleotide sequence) The truncated lysostaphin (the mature lysostaphin coding sequence lacking the two 5′-most Ala codons of the wild-type mature protein) was then amplified by PCR using pLss1C as a template. The resulting amplified product contained the coding region for truncated lysostaphin, a 5′ TG sequence for translation initiation (together with the last T of the cut restriction site Scal, a TTG codon is constituted that can serve as an initiation codon in Lactococci), and a 3′ Xbal restriction site for cloning purposes. This PCR product was then digested with Xbal and ligated into pNZ8148Scal cut with Scal and Xbal, resulting in plasmid pLss12C (not shown). Finally, the chloramphenicol resistance gene was exchanged for the lacF gene (originating from plasmid pNZ8148F-1, not shown) using the restriction sites Sall and NspV, resulting in plasmid pLss12F. Restriction maps of pRN5550, pNZ8148Scal, pLss1C, and pLss12F can be found respectively in FIGS. 7A-D.

In pLss12F, the theoretical N-terminal amino acid sequence for truncated lysostaphin is f-Met-Thr-His-Glu. In many cases, the f-Met is cleaved off by a dedicated peptidase. N-terminal amino acid sequencing results to date suggest that indeed this 5′ f-Met is sometimes cleaved off.

pLss12F was isolated and purified by standard methods as described in Sambrook, J., et al. (17).

Transformation of host cells with the expression plasmid:

L. lactis subsp. cremoris NZ3900, a derivative of strain MG1363 (pepN:: nisRK, lac⁺lacF, prophage-cured), was transformed with pLss12F and plated on Elliker agar supplemented with 1% lactose. Transformants were selected on the basis of their ability to grow on this agar and to ferment lactose. After screening for correct plasmid retention, the resulting strain was called NZ3900 (pLss12F).

Initiation of Seed Train in Laboratory:

Each of two vials of NZ3900 (pLss12F) Initial Cell sank Working Stock were inoculated into 300 mL of Soy Peptone/Yeast Extract Medium and incubated overnight (16 to 24 hours) at 30±3° C.

Seed Train—30 L Fermentation:

The overnight cultures were used to inoculate 30 L of Soy Peptone/Yeast Extract Medium in a Chemap 75 fermentor. The fermentor ran overnight (16 to 20 hours) at 30±3° C.

Production—3000 L Fermentation:

Three thousand liters of Soy Peptone/yeast Extract Medium in a 4000 liter fermentor were inoculated with the 30 liter seed culture. Inclusion of approximately 3.4 μm zinc in the fermentation media may maintain or increase lysostaphin activity, since zinc is required for lysostaphin activity. The fermentation proceeded at 30±2° C. and pH 6.5±0.2; pH was regulated with 5 M NaOH. Induction using Nisin at a concentration of 10 ng/mL was performed when the culture reached an OD600 of approximately 1.0. Lysostaphin production proceeded for 6 hours before the process was stopped by cooling the fermentor content down to below 10° C.

Concentration of Biomass:

The entire L. lactis biomass of the fermentation was concentrated on a 3.8 m² 0.8 μm membrane by microfiltration. The cells were then diafiltered (200%) against water.

Release of Lysostaphin—Homogenization:

The concentrated cells were homogenized 3 times in 150 mM NaCl/25 mM Na phosphate, pH 7 buffer in an APV homogeniser at <10° C., releasing lysostaphin into the homogenate.

Removal of Cellular Debris:

Cellular debris was removed from the liquid homogenate by ultrafiltration using a 500-750 kDa ultrafiltration membrane. Following ultrafiltration, the lysate was in approximately 150 mM NaCl, 25 mM sodium phosphate pH 7, and was diluted 1:2 with water. Dilution reduces the solution conductivity below 10 mS/cm to allow efficient lysostaphin capture on the cationic chromatography resin.

Capture—SP-Sepharose Chromatography:

The permeate was adjusted to pH 7.2 with 0.4 M Na₂HPO₄, filtered on a Sartobran P filter capsule, and purified on SP-sepharose in a BPG3000 column that had been equilibrated with two column volumes of 50 mM Sodium Phosphate, pH 7.5. After washing with three column volumes of this buffer, the lysostaphin was eluted with two column volumes of 25 mM Sodium Phosphate, 0.25 M NaCl, pH 7.5. All eluted fractions containing lysostaphin were collected and stored at <−20° C.

Dilution/Filtration:

Approximately twelve liters of SP-Eluate was removed from the freezer and allowed to thaw at room temperature for approximately 18 hours. The material was pooled in a single container and was then mixed gently to create a homogeneous solution. Twelve liters of Q Equilibration Buffer (0.05 M Tris, pH 7) was added to the SP-Eluate pool to bring the conductivity of the solution to the target range of 8-16 mS/cm. The mixture was run through a 0.45 μm Sartoclean filter unit into a 50 liter Stedim bag to create the Filtered SP-Eluate Pool.

Q-Sepharose Purification:

The Filtered SP-Eluate Pool was purified on a previously qualified Q-Sepharose FF Chromatography column that had been regenerated with approximately two column volumes of Q Regeneration Buffer (0.05 M Tris, 0.04 M NaOH, 1 N NaCl) and was subsequently equilibrated with approximately three column volumes of Q Equilibration Buffer (0.05 M Tris, 0.04 M NaOH). The Filtered SP-Eluate Pool was loaded onto the column until 6 liters of Q Flow Through (Q F-T 1) had been collected in a waste container. The remaining pooled material was loaded onto the column and the remaining flow through (Q F-T 2) was collected in a lined tank. Approximately twelve liters of Q Equilibration Buffer was then run onto the column. A total of 29.38 liters of Q F-T 2 material was collected.

Q Flow Through—Dilution/Filtration:

Ammonium Sulfate (3M (NH₄)₂SO₄) was mixed into the solution [volume of ammonium sulfate added =(Q F-T 2)÷2.75] over the course of 15 minutes; the pH of the solution was then adjusted to 6-7 using Phosphoric Acid. The Q F-T 2 was filtered (0.45 μM) into a single bag and stored overnight at 2-8° C. (Filtered Q Eluate).

Phenyl Sepharose Purification:

The following day, the Filtered Q Eluate was purified on a previously qualified Phenyl-Sepharose HP column that had been equilibrated with approximately two column volumes of Phenyl Equilibration Buffer (0.05 M NaH₂PO₄, 0.8 M (NH₄)₂SO4,).

After loading the Filtered Q Eluate on the column, it was rinsed with approximately two column volumes of Phenyl Rinse Buffer (0.05 M NaH₂PO₄, 0.5 M (NH₄)₂SO₄). Purified lysostaphin API was eluted with Phenyl Elution Buffer (0.05 M NaH₂PO₄, 0.25 M (NH₄)₂SO₄). Approximately 51 liters of Phenyl Eluate was collected (OD₂₈₀ of the eluate was >0.2). The material was stored at ambient temperature overnight.

Ultrafiltration/Diafiltration:

The following day, a sanitized Millipore Pellicon Ultrafiltration Assembly equipped with a 20 ft² 5 KD molecular weight cut off (MWCO) Pellicon Cassette was equilibrated with 3 liters of Phenyl Regeneration Buffer and drained. The Phenyl Eluate was concentrated by ultrafiltration until a target lysostaphin concentration (as determined by OD₂₈₀ measurement) of approximately 25 mg/mL had been achieved. The concentrated Phenyl Eluate was then diafiltered against WFl. The permeate was collected in a lined tank until the conductance of the material was reduced to 0.5-0.6 mS/cm. The protein concentration of the Diafiltered Concentrate was determined by OD₂₈₀ and additional permeate was collected to obtain a target lysostaphin concentration of approximately 25 mg/mL. The Pellicon apparatus was rinsed and drained and the rinse volume was added to the collected permeate (Diafiltered Phenyl Eluate, Formulated Concentrate).

Filtration:

The Formulated Concentrate was filtered through a Millipak 200 unit (0.2 μM filter) into two separate Stedim 5 liter bags (Bag 1 and Bag 2) to create Bulk APl. Samples were collected from each bag and were submitted for analytical, bioburden, and endotoxin testing.

Bulk APl (Active Pharmaceutical Ingredient):

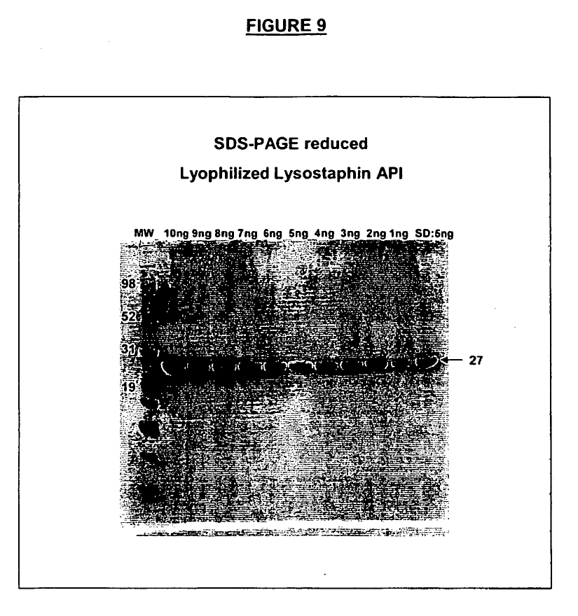

The Bulk APl, Diafiltered Q- and Phenyl Sepharose purified lysostaphin was stored at 2-8° C. FIG. 9 shows an example of lysostaphin APl on an SDS-PAGE reducing gel.

### Example 6

Comparison Of Homogenous Mature and Homogenous Truncated Lysostaphin By Killing Assay With Different Concentrations of Lysostaphin

Homogenous mature (AATHE) and truncated lysostaphins (THE), prepared essentially as described above, were compared in a standard potency assay as follows:

Preparation of Lysostaphin Working Stocks for Assay:

Approximately 1 milligram of lysostaphin powders were dissolved in 1 ml each of PBS to create stock samples. Stock samples were diluted 1:5 and 1:10 (200 μl in 800 μl and 100 μl in 900 μl). The A₂₈₀ of the 1:5 and 1:10 dilutions was read on a BioRad SmartSpec 3000 spectrophotometer blanked with PBS. Concentrations (in mg/ml) of the stock solutions were determined by multiplying the absorbance of each dilution by 0.49 (the reciprocal of the extinction coefficient) and the dilution factor (5 or 10) to determine the concentration in mg/ml. Stock solutions were only used where the determined concentrations for each dilution was within 10% of each other. Stock solutions were further diluted to 5 to 6 ng/μl working stocks with PBS and kept on ice until used. Aliquots of working stocks were stored at −70° C.

Bacterial Preparation:

Frozen stocks of S. aureus type 5 ATCC 49521 were streaked on blood agar and incubated overnight. Three colonies were transferred to 1 ml of DIFCO tyrptic soy broth and incubated overnight at 37° C. w/ shaking to form a bacterial stock. The bacterial stock was normalized with PBS to an OD₆₅₀ of 0.750 to 0.790. 1:10 serial dilutions of the normalized stock were made in PBS. Actual starting CFUs were determined by mixing 350 ul of normalized stock with 650 μl of PBS and plating 100 μl on blood agar.

Lysostaphin Dilutions:

Three independent sets of dilutions were made for each lysostaphin (18 tubes per sample, 6 tubes per set, 3 tubes per series). Each set contained two dilution series of 48, 24, 12 ng/ml lysostaphin and 32, 16, 8 ng/ml lysostaphin. This resulted in 18 tubes for each lysostaphin with 650 μl per tube for each sample divided into 3 sets per sample (each set consists of 48, 32, 24, 16, 12, and 8 ng).

Performance of assay:

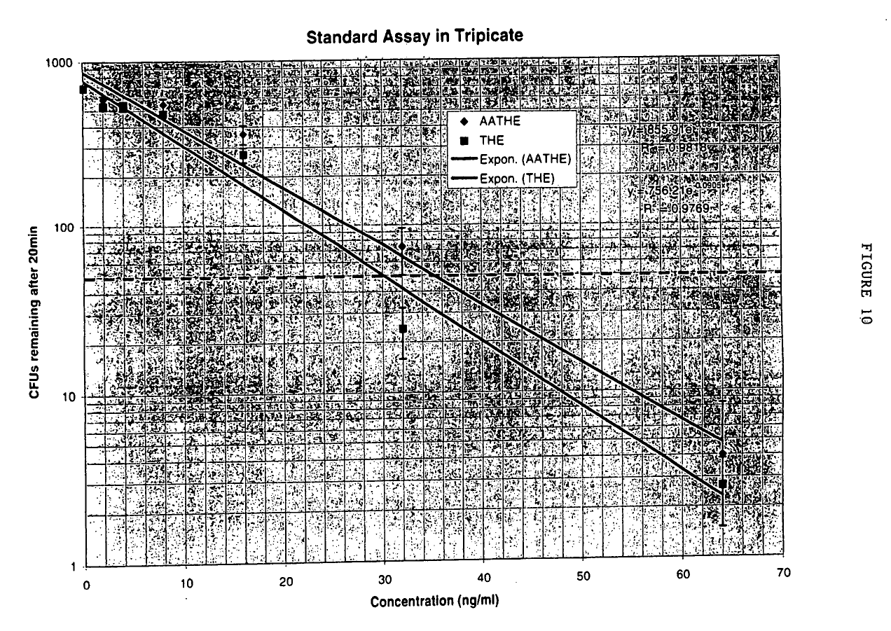

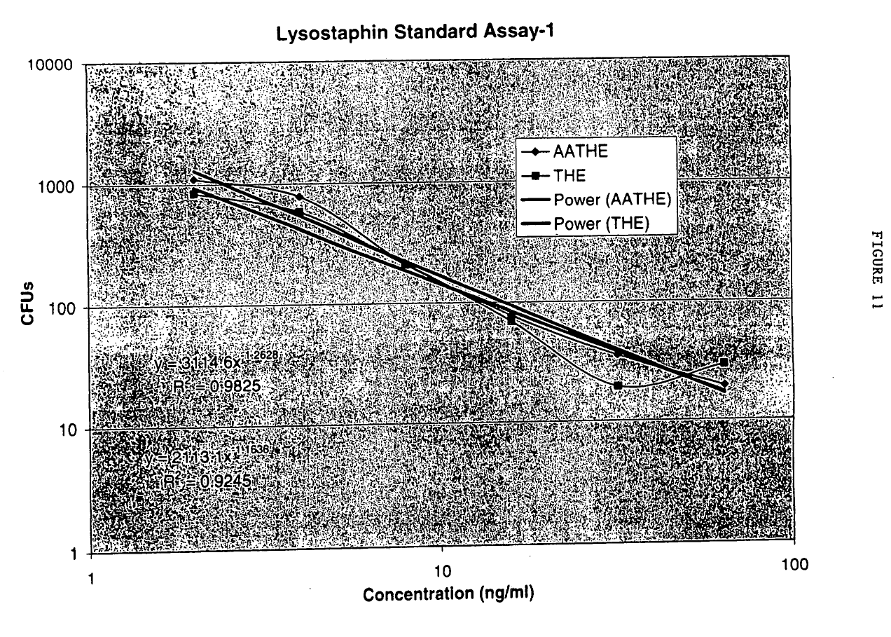

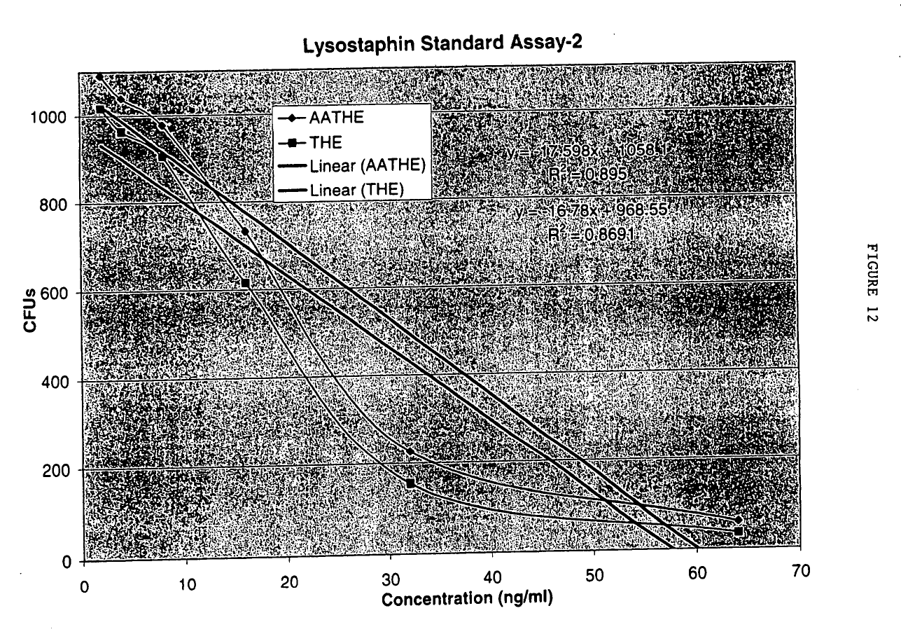

To start the reaction, 350 μl of working stock bacteria was added to each of the 18 tubes in the sample set and mixed by vortexing. The samples were incubated for 20 minutes at room temperature with additional mixing at 10 minutes and just prior to plating. After 20 minutes, 100 μl samples from each tube are plated onto blood agar plates and incubated overnight at 37° C. Colonies were enumerated and the results plotted. The results of three such assays, depicted in FIGS. 10-12, demonstrate the generally greater bacteriocidal activity of the truncated lyphostaphin (THE . . . ) as compared to the mature form (AATHE . . . ) over a wide concentration range.

### Example 7

Comparison Of Homogenous Mature and Homogenous Truncated Lysostaphin By Time Course Killinci Assay On Cultures Grown in Whole Blood

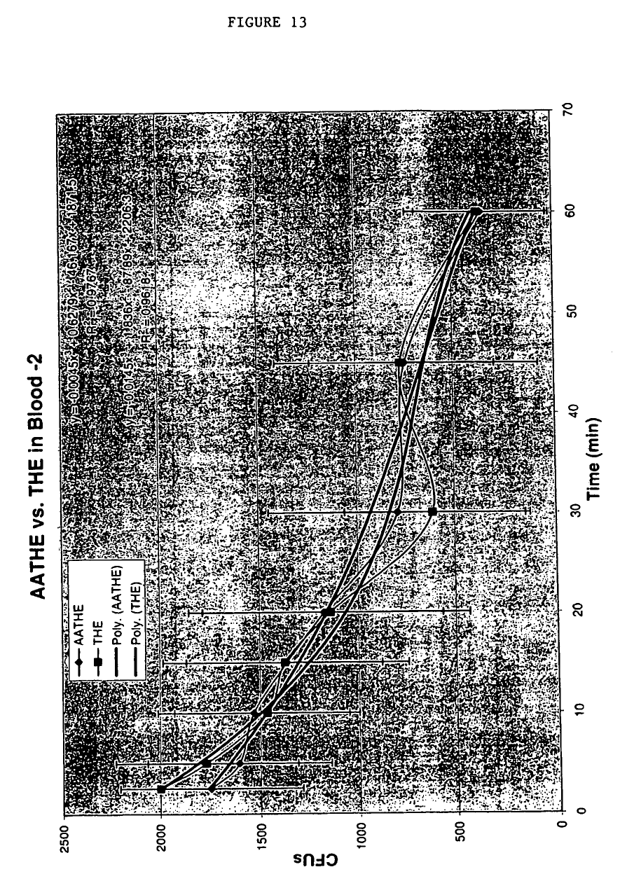

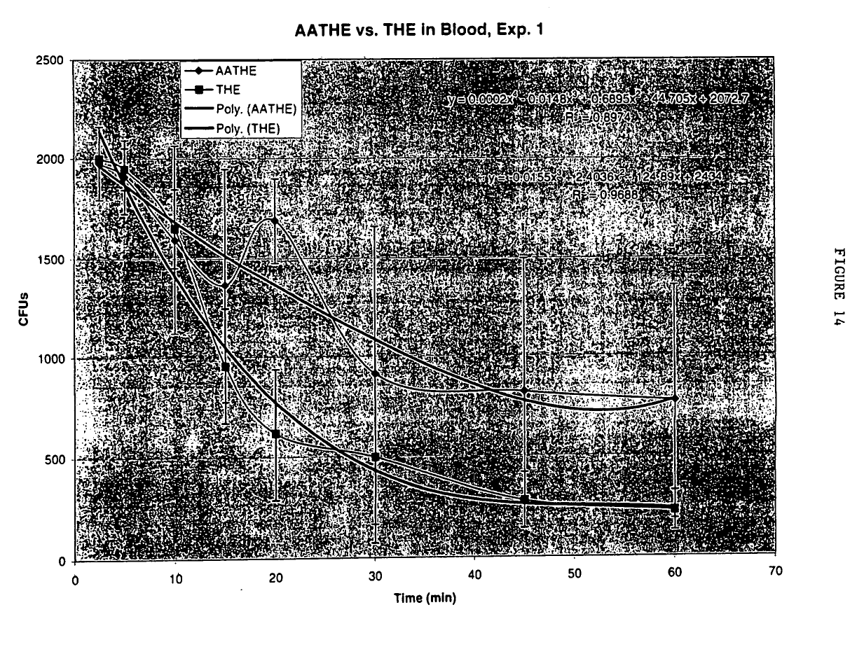

Lysostaphin is generally less active in whole blood than in a buffer. To further examine the enhanced activity of truncated lysostaphin versus full length lysostaphin, homogenous mature and truncated lysostaphins were compared in a time course killing assay in whole blood as follows. Cultures of S. aureus were grown overnight in DIFCO tryptic soy broth. 50 μl aliquots of the overnight S. aureus cultures were used to inoculate six tubes containing 1 ml of fresh human blood each. Inoculated bloods were grown overnight at 37° C. with shaking and pooled the next day. 1 ml aliquots of the pooled blood cultures were mixed with 1 microgram of homogeneous mature (AATHE) or truncated (THE) lysostaphin and incubated at room temperature. At specified time points, 100 μl samples of each were diluted 1:100 in PBS. 100 μl of each PBS dilution was plated on a blood agar plate and cultured overnight at 37° C. to allow colony growth. Colonies were enumerated and the results plotted in FIGS. 13 and 14. These results indicate that the greater bacteriocidal activity of truncated lyphostaphin (THE . . . ) as compared with the mature form (AATHE . . . ) appears more pronounced at longer incubation times.

### Example 8

Comparison Of Homogenous Mature and Homogenous Truncated Lysostaphin By Optical Density Drop Assay

Homogenous mature and truncated lysostaphins were compared in a time course turbidity drop assay as follows.

Test sample preparation:

Lysostaphin stock solutions were prepared in PBS as above and diluted in PBS to approximately 0.1 mg/ml working stocks.

Preparation of bacteria for assay:

3-5 ml cultures of Staphylococcus carnosus were grown overnight in DIFCO tryptic soy broth from a blood agar plate <1 week-old. 1.5 milliliters of overnight S. carnosus culture was pelleted by microfuged at maximum rpm for 5 minutes. The bacterial pellet was washed with an equal volume of PBS and repelleted. The washed pellet was resuspended in 3 ml PBS diluted with PBS to a final OD650 of 1.55+/−0.04. The diluted bacteria were stored on ice until use.

Performance of assay:

The drop in turbidity of the resuspended S. carnosus was followed on a BioRad SmartSpec 3000 blanked against PBS and set for “Kinetics” assay, using as parameters; 650 nM, 900 sec total duration, 30 sec interval, and no background subtraction. For each lysostaphin preparation, 500 ul of the ˜1.55 absorbance bacteria were pipetted into a fresh cuvette and allowed to warm to room temperature. A zero reading was determined, followed rapidly by the addition and mixing of 1 ug (10 ul) of lysostaphin (either AATHE or THE). The OD650 of each mixture was determined over a period of sixty minutes and the lysostaphin activity determined according to the following formula:

$$\frac{( Time ( min ) to {OD}_{650} of reference {lysostaphin }^{*} ug added )}{( Time ( min ) to {OD}_{650} of sample {lysostaphine }^{*} ug added )} * 100 = Units \text{/} ug$$

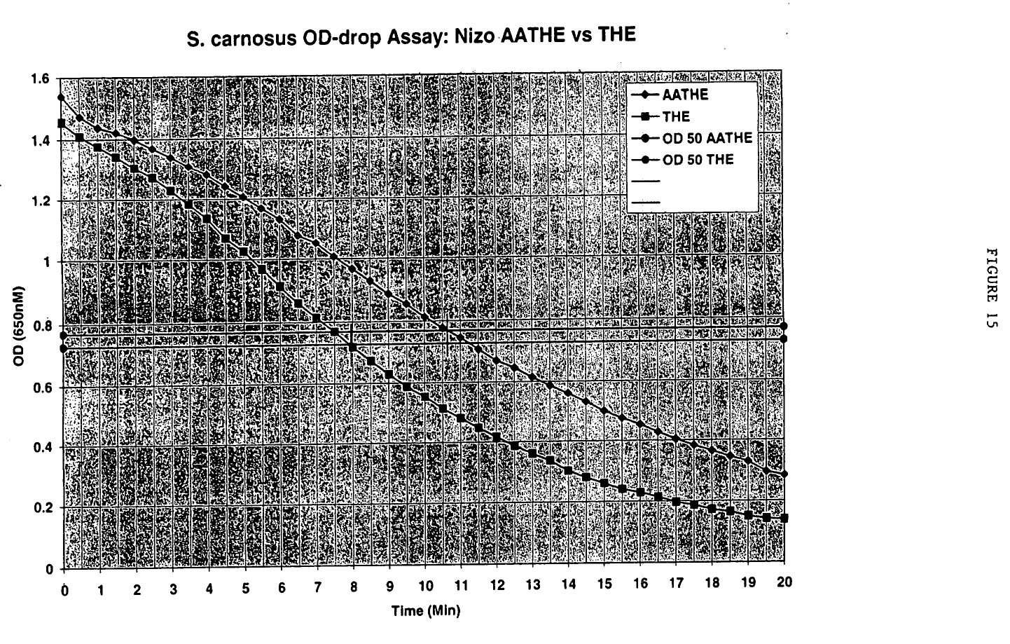

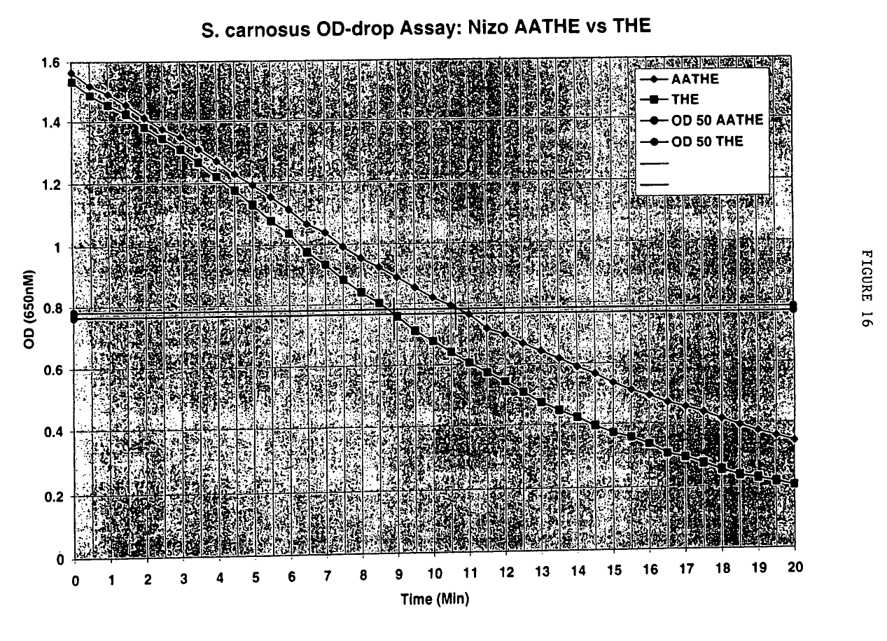

The results of two optical density drop assays, shown in FIGS. 15 and 16, illustrate the more rapid bacteriocidal activity of the truncated lyphostaphin (THE . . . ) than the mature form (AATHE . . . ). The inventors note that this assay was terminated at 60 minutes. In vivo, where incubation times are measured in hours and days, activity of truncated lysostaphin may be even more markedly superior than that of the mature form.

### CONCLUSION

Thus, Examples 1 and 2 describe the design and production of recombinant DNA molecules that express a homogenous form of lysostaphin. These plasmids allow for either intracellular expression of lysostaphin or for secretion of lysostaphin extracellularly. Example 3 shows that these plasmids, when transformed into bacterial host cells, do express lysostaphin and that this lysostaphin is enzymatically active. Example 4 demonstrates that the truncated form of lysostaphin exhibited greater antistaphylococcal activity than its “mature” full length counterpart. Example 5 describes a bulk purification protocol for recombinant lysostaphin. Examples 6,7, and 8 demonstrate that the truncated THE form of lysostaphin has superior anti-staphylococcal activity over the mature AATHE form of lysostaphin.

One of skill in the art would realize that the recombinant DNA molecules of the invention are not limited to only those described in the above examples. One of ordinary skill would know that alternate host cells may be used for expression. Such a person would also know to substitute genetic elements on the recombinant DNA molecule, such as promoters and replication origins, for alternate sequences that achieve the same function in that alternate host cell. These alternate sequences are well known to those of ordinary skill in the art. Further, the instant invention is not limited to lysostatphin that is purified by the above-described methods. One of ordinary skill in the art would be able to use alternate methods of protein purification to achieve the same ends of the invention.

The following publications are hereby specifically incorporated by reference:

1. Chang, F. Y., N. Singh, T. Gayowski, S. D. Drenning, M. M. Wagener and I. R. Marino. 1998. Staphylococcus aureus nasal colonization and association with infections in liver transplant recipients. Transplantation 65:1169-1172.

2. Chapoutot, C., G.-P. Pageaux, P. F. Perrigault, Z. Joomaye, P. Perney, H. Jean-Pierre, O. Jouquet, P. Blanc and D. Larrey. 1999. Staphylococcus aureus nasal carriage in 104 cirrhotic and control patients A prospective study. J. Hepatol. 30:249-253.

3. Fierobe, L., D. Decre, C. Muller, J.-C. Lucet, J.-P. Marmuse, J. Mantz and J.-M. Demonts. 1999. Methicillin-resistant Staphylococcus aureus as a causative agent of postoperative intra-abdominal infection: relation to nasal colonization. Clin. Infect. Dis. 29:1231-1238.

4. Frebourg, N., B. Cauliez and J.-F. Lerneland. 1999. Evidence for nasal carriage of methicillin-resistant staphylococci colonizing intravascular devices. J. Clin. Micro. 37:1182-1185.

5. Heinrich, P., R. Rosenstein, M. Bohmer, P. Sonner, and F. Gotz 1987. The molecular organization of the lysostaphin gene and its sequences repeated in tandem. Mol. Gen. Genet. 209:563-9.

6. Kline S A, J. de la Harpe , and P. Blackburn. 1994. A colorimetric microtiter plate assay for lysostaphin using a hexaglycine substrate. Anal Biochem. 217:329-31.

7. Kluytmans, J., A. van Belkum and H. Verbrugh. 1997. Nasal carriage of Staphylococcus aureus: epidemiology, underlying mechanisms, and associated risks. Clin. Micro. Rev. 10:505-520.

8. Kluytmans, J. A., J. W. Mouton, M. VandenBergh, M.-J. Manders, A. Maat, J. Wagenvoort, M. Michel and H. Verbrugh. 1996. Reduction of surgical-site infections in cardiothoracic surgery byelimination of nasal carriage of Staphylococcus aureus. Infect. Control.Hosp. Epidem. 17:780-785.

9. Martin, J., F. Perdreau-Remington, M. Kartalija, O. Pasi, M. Webb, J. Gerberding, H. Chambers, M. Tauber and B. Lee. 1999. A randomized clinical trial of mupirocin in the eradication of Staphylococcus aureus nasal carriage in human immunodeficiency virus disease. J. Infect. Dis. 180:896-899.

10. Merkus, F. W., J. C. Verhoef, N. G. Schipper, and E. Marttin. 1999. Cyclodextrins in nasal drug delivery. Advan. Drug Deliv. Rev. 36:41-57.

11. Nakamura, K. et al. 1999. Uptake and Release of Budesonide from Mucoadhesive, pH-sensitive Copolymers and Their Application to Nasal Delivery. J. Control. Release 61:329-335.

12. Natsume, H., S. Iwata, K. Ohtak, M. Miyamoto, M. Yamaguchi, K. Hosoya, and D. Kobayashi. 1999. Screening of cationic compounds as an absorption enhancer for nasal drug delivery. lnt. J. Pharma. 185:1-12.

13. Nguyen, M. H., C. Kauffman, R. Goodman, C. Squier, R. Arbeit, N. Singh, M. Wagener and V. Yu. 1999. Nasal carriage of and infection with Staphylococcus aureus in HIV-infected patients. Ann. Int. Med. 130:221-225.

14. Ramkissoon-Ganorkar, C. et al. 1999. Modulating insulin-release profile from pH/thermosensivite polymeric beads through polymer molecular weight. J. Contr. Release 59:287-298.

15. Remington's Pharmaceutical Sciences, 18th Edition (A. Gennaro, ed., Mack Pub., Easton, Pa., 1990).

16. Ribeiro, A. J. et al. 1999. Microencapsulations of lipophilic drugs in chitosan-coated alginate microspheres. Int. J. Pharm. 187:115-123.

17. Sambrook, J., Fritsch, E. F., and Maniatis, T. (1989). Molecular cloning: a laboratory manual, 2nd Edition., Cold Spring Harbour Laboratory Press, Cold Spring Harbour.

18. Schwab, U. E., A. E. Wold, J. L. Carson, M. W. Leigh, P.-W. Cheng, P. H. Gilligan and T. F. Boat. 1993. Increased adherence of Staphylococcus aureus from cystic fibrosis lungs to airway epithelial cells. Am. Rev. Respir. Dis. 148:365-369.

19. Soane, R.J. et al. 1999. Evaluation of the clearance characteristics of bioadhesive systems in humans. Int. J. Pharm. 178:55-65.

20. Soto, N., A. Vaghjimal, A. Stahl-Avicolli, J. Protic, L. Lutwick and E. Chapnick. 1999. Bacitracin versus mupirocin for Staphylococcus aureus nasal colonization. Infect. Cont. Hosp. Epidem. 20:351-353.

21. Suzuki, Y. and Y. Makino. 1999. Mucosal drug delivery using cellulose derivative as a functional polymer. J. Control. Release. 62:101-107.

22. Takenaga, M., Y. Sirizawa, Y. Azechi, A. Ochiai, Y. Kosaka, R. Igarashi, and Y. Mizushima. 1998. Microparticle resins as a potential nasal drug delivery system for insulin. J. Control. Release. 52:81-87.

23. VadenBergh, M., E. Yzerman, A. Van Belkum, H. Boelens, M. Simmons and H. Verbrugh. 1999. Follow-up of Staphylococcus aureus nasal carriage after 8 years: redefining the persistent carrier state. J. Clin. Micro. 37:3133-3140.

24. White, A. and J. Smith. 1963. Nasal reservoir as the source of extranasal staphylococci. Antimicrob. Agent. Chem. 3:679-683.

25. Yano, M., Y. Doki, M. Inoue, T. Tsujinaka, H. Shiozaki and M. Monden. 2000. Preoperative intranasal mupirocin ointment significantly reduces postoperative infection with Staphylococcus aureus in patients undergoing upper gastrointestinal surgery. Surg. Today (Japan). 30:16-21.

26. Yu, V. L., A. Goetz, M. Wagener, P. B. Smith, J. D. Rihs, J. Hanchett and J. J. Zuravleff. 1986. Staphylococcus aureus nasal carriage and infection in patients on hemodialysis. New Engl. J. Med. 315:91-96.

27. Zygmunt, W. A. and P. A. Tavormina.t 1972. Lysostaphin: Model for a Specific Enzymatic Approach to Infectious Disease. Prog. Drug Res. 16:309-333.

28. Climo, M. W., R. L. Patron, B. P. Goldstein, and G. L. Archer. 1998. Lysostaphin treatment of experimental methicillin-resistant Staphylococcus aureus aortic valve endocarditis. Antimicrob. Agents Chemother. 42:1355-1360.

29. Kokai-Kun, J., T. Chanturiya, and J. Mond. 2002. Lysostaphin as a therapy for systemic Staphylococcus aureus infection. Presented at the American Society for Microbiology 102nd General Meeting, Salt Lake City, Utah.

30. Other embodiments of the invention will be apparent to those skilled in the art from consideration of the specification and practice of the invention disclosed herein. It is intended that the specification and examples be considered as exemplary only, with a true scope and spirit of the invention being indicated by the following claims

1. An isolated DNA molecule comprising a promoter region operably linked to a DNA sequence encoding a truncated lysostaphin protein.

2. The isolated DNA molecule of claim 1; wherein expression of the truncated lysostaphin results in a collection of protein molecules having N-terminal amino acid sequences selected from one or both of “MTHE . . . ” and “THE . . . ”.

3. The isolated DNA molecule of claim 1 or claim 2, further comprising a signal peptide operably linked to the DNA sequence encoding a truncated lysostaphin protein; wherein the signal peptide directs the secretion of the truncated lysostaphin protein.

4. The isolated DNA molecule of claims 1-3, wherein the truncated lysostaphin protein comprises amino acids 2-245 of SEQ ID NO: 7, or an active lysostaphin variant thereof.

5. A host cell transformed with the isolated DNA molecule of claims 1-4.

6. The host cell of claim 5, wherein said host cell is E. coli, L. lactis, or B. sphaericus.

7. A method of producing recombinant homogenous truncated lysostaphin comprising:
a. culturing the host cell of claim 5 or claim 6;
b. inducing expression of truncated lysostaphin;
c. lysing the host cells; and
d. isolating truncated homogenous lysostaphin from the lysed host cells.

8. A method of producing recombinant homogenous truncated lysostaphin comprising:
a. culturing the host cell of claim 5 or claim 6;
b. inducing expression of truncated lysostaphin;
c. concentrating the host cell media; and isolating truncated homogenous lysostaphin from the concentrated host cell media.

9. A homogenous truncated lysostaphin.

10. The lysostaphin of claim 9 comprising lysostaphin molecules having N-terminal amino acid sequences selected from one or both of “MTHE . . . ” and “THE . . . ”.

11. The lysostaphin of claim 10, wherein at least 50% of said molecules have N-terminal amino acid sequences consisting the amino acids “THE . . . ”.

12. The lysostaphin of claim 11, wherein substantially all of said molecules have N-terminal amino acid sequences consisting the amino acids “THE . . . ”.

13. A medicament composition comprising:
a. the homogenous truncated lysostaphin of any of claims 9-12; and
b. a pharmaceutically acceptable carrier.
